(19) United States
(12) Patent Application Publication
(10) Pub. No.: US 2007/0232697 A1
(43) Pub. Date: Oct. 4, 2007

(54) Method for preparing free base compositions and formulations thereof

(75) Inventors: Jianbo Xie, Davie, FL (US); David Barman, North Miami Beach, FL (US); Mongkol Sriwongjanya, Pembroke Pines, FL (US); Sam Yuk, Boca Raton, FL (US)

Correspondence Address: DAVIDSON, DAVIDSON & KAPPEL, LLC, 485 SEVENTH AVENUE, 14TH FLOOR, NEW YORK, NY, 10018, US

(73) Assignee: Andrx Labs LLC, Davie, FL (US)

(21) Appl. No.: 11/391,918

(22) Filed: Mar. 29, 2006

(51) Int. Cl. A61K 31/205; A61K 31/138

(52) U.S. Cl. 514/554; 514/651

(57) ABSTRACT

Disclosed is a process for preparing a free base composition of a pharmaceutical compound comprising combining an acid addition salt of a pharmaceutical compound, at least one solvent and a sufficient amount of at least one base to convert the acid addition salt of the pharmaceutical to a free base of the pharmaceutical, to form a free base composition of the pharmaceutical compound.

### FIELD OF THE INVENTION

The present invention is related to a process for conversion of an acid addition salt of a pharmaceutical to the free base form of the pharmaceutical and formulations for delivery of the free base composition. In preferred embodiments, the process comprises conversion of an acid addition salt of metoprolol to a free base metoprolol composition.

### BACKGROUND OF THE INVENTION

It is also a common goal in the pharmaceutical arts to provide formulations of active ingredients or analogs of active ingredients which possess increased bioavailability when administered to patients in need of therapy.

It is another common goal to provide formulations and analogs of active agents which have a slower dissolution rate which can provide improved controlled release formulations of the active ingredient.

Metoprolol is a β-blocker known to be effective in treatment of hypertension, angina pectoris, myocardial infarction and migraines.

U.S. Pat. No. 5,081,154 to Appelgren et al. describes metoprolol succinate and pharmaceutical formulations containing this salt form.

There is a need for processes for conversion of acid addition salts of pharmaceuticals to the free base form. There is a further need for preparing active agent formulations, e.g., metoprolol formulations, which have equal or greater bioavailability and prolonged release properties as compared to formulations containing an acid addition salt of the pharmaceutically active agent.

### OBJECTS AND SUMMARY OF THE INVENTION

It is an object of the invention to provide a process for preparing a free base composition of a pharmaceutically active agent.

It is an object of certain embodiments of the invention to provide a process for preparing a free base composition of metoprolol.

It is an object of certain embodiments of the invention to provide a process for preparing a free base composition of a pharmaceutical compound comprising combining an acid addition salt of a pharmaceutical compound, at least one solvent and a sufficient amount of a base to convert the acid addition salt of the pharmaceutical to a free base of the pharmaceutical, to form a free base composition of the pharmaceutical compound.

It is an object of certain embodiments of the invention to provide a process for preparing a free base composition of a pharmaceutical compound comprising (a) dissolving an acid addition salt of a pharmaceutical compound in at least one solvent; and (b) adding at least one base in a sufficient amount to substantially convert the acid addition salt of the pharmaceutical to a free base of the pharmaceutical, to form a free base composition of the pharmaceutical compound.

It is an object of certain embodiments of the invention to provide a process for preparing a metoprolol free base composition comprising combining an acid addition salt of metoprolol, at least one solvent and a sufficient amount of at least one base to convert the acid addition salt of the metoprolol to free base metoprolol, to form a free base metoprolol composition.

It is an object of certain embodiments of the invention to provide an oral solid dosage form comprising an effective amount of the free base composition prepared according to the processes disclosed herein.

It is an object of certain embodiments of the invention to provide an oral solid dosage form comprising an effective amount of a metoprolol free base composition prepared according to the processes disclosed herein.

It is an object of certain embodiments of the invention to provide an oral solid dosage form comprising an effective amount of an organic free base composition prepared according to the processes disclosed herein, which has increased bioavailability as compared to formulations comprising an equivalent amount of a pharmaceutical salt of the organic free base.

It is an object of certain embodiments of the invention to provide an oral solid dosage form comprising an effective amount of a metoprolol free base composition prepared according to the processes disclosed herein, which has increased bioavailability as compared to formulations comprising an equivalent amount of metoprolol salt.

It is an object of certain embodiments of the invention to provide an oral solid dosage form comprising an effective amount of an organic free base composition prepared according to the processes disclosed herein, which has equivalent bioavailability as compared to formulations comprising an equivalent amount of a pharmaceutical salt of the organic free base.

It is an object of certain embodiments of the invention to provide an oral solid dosage form comprising an effective amount of a metoprolol free base composition prepared according to the processes disclosed herein, which has equivalent bioavailability as compared to formulations comprising an equivalent amount of metoprolol salt.

It is an object of certain embodiments of the invention to provide a method of increasing the plasma level of a pharmaceutical compound comprising combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound; and incorporating the free base composition into a dosage form in a therapeutically effective amount, wherein the maximum plasma concentration of the free base composition is greater than the maximum plasma concentration achieved by an equivalent amount of the acid addition salt of the pharmaceutical compound.

It is an object of certain embodiments of the invention to provide a method of increasing the bioavailability of a pharmaceutical compound comprising combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound; and incorporating the free base composition into a dosage form in a therapeutically effective amount, wherein the bioavailability of the free base composition is greater than the bioavailability achieved by an equivalent amount of the acid addition salt of the pharmaceutical compound.

It is an object of certain embodiments of the invention to provide a method of utilizing reduced amounts of a pharmaceutical compound comprising combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound; and incorporating the free base composition into a dosage form in an amount to achieve a therapeutically effective maximum plasma concentration of the free base composition, wherein the amount of the free base composition is less than an amount of the acid addition salt of the pharmaceutical compound to achieve the same maximum plasma concentration.

It is an object of certain embodiments of the invention to provide a method of utilizing reduced amounts of a pharmaceutical compound comprising combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound; and incorporating the free base composition into a dosage form in an amount to achieve a therapeutically effective AUC measurement of the free base composition, wherein the amount of the free base composition is less than an amount of the acid addition salt of the pharmaceutical compound to achieve the same AUC.

It is an object of certain embodiments of the invention to provide a method of treating a patient comprising administering a free base pharmaceutically active agent in an amount to achieve a therapeutically effective maximum plasma concentration of the active agent, wherein the amount of the free base active agent is less than an amount of an acid addition salt of the active agent to achieve the same maximum plasma concentration.

It is an object of certain embodiments of the invention to provide a method of treating a patient comprising administering a free base pharmaceutically active agent in an amount to achieve a therapeutically effective AUC measurement of the active agent, wherein the amount of the free base active agent is less than an amount of an acid addition salt of the active agent to achieve the same AUC measurement

It is an object of certain embodiments of the invention to provide a dosage form which provides a bioavailability which is from 80% to 125% of the corresponding value of a reference standard, e.g., Toprol XL®.

It is an object of certain embodiments of the invention to provide a dosage form which provides a maximum plasma concentration which is from 80% to 125% of the corresponding value of a reference standard, e.g., Toprol XL®.

It is an object of certain embodiments of the invention to provide a dosage form which provides a time to maximum plasma concentration which is from 80% to 125% of the corresponding value of a reference standard, e.g., Toprol XL®.

The free base composition of the present invention can be combined with a pharmaceutically acceptable carrier, and the resulting mixture may be processed to obtain a free base composition tablet. In preferred embodiments, the free base composition is a solution or suspension which is sprayed onto the carrier.

In one embodiment of the invention, the pharmaceutically acceptable carrier comprises a plurality of particles of a material such as, for example, anhydrous lactose or microcrystalline cellulose. A granulate is formed by spraying the free base composition onto the carrier. Additional processing steps may then be undertaken to prepare a uniform granulate suitable for formulating into tablets. Sufficient quantities of pharmaceutically necessary tableting excipients may then be admixed with the free base granulate, and the resulting mixture may be compressed into tablets.

In other embodiments, the free base composition can be combined, e.g., granulated with, a hydrogel forming material. The hydrogel material can include but is not limited to a hydroxyalkylcellulose (e.g., hydroxypropyl cellulose, hydroxypropylmethyl cellulose); polyalkylene oxide having a weight average molecular weight of 100,000 to 6,000,000 (e.g., poly(ethylene) oxide, poly(methylene oxide), poly(butylene oxide), and poly(hexylene oxide); poly(hydroxy alkyl methacrylate) having a molecular weight of from 25,000 to 5,000,000; poly(vinyl)alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a hydrogel forming copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anyhydride in the copolymer; Carbopol® acidic carboxy polymers having a molecular weight of 450,000 to 4,000,000; Cyanamer® polyacrylamides; cross-linked water swellable indenemaleic anhydride polymers; Goodrite® polyacrylic acid having a molecular weight of 80,000 to 200,000; starch graft copolymers; Aqua-Keeps® acrylate polymer polysaccharides composed of condensed glucose units such as diester cross-linked polyglucan and the like. Other polymers which form hydrogels are described in U.S. Pat. Nos. 3,865,108; 4,002,173 and 4,207,893 all of which are incorporated by reference. Mixtures of the aforementioned pharmaceutically acceptable polymers may also be used.

The free base composition tablets may be coated with an enteric coating to produce delayed-release tablets. Optionally, a seal coating may also be applied to the tablets before the enteric coating is provided. The enteric coated free base composition tablets may be further overcoated with a film-coating.

In accordance with the invention, the pharmaceutically acceptable carrier may comprise a plurality of inert beads, for example, sugar beads or nonpareil seeds. The free base composition can be sprayed onto the inert beads to produce free base composition coated beads, which can then be formulated into solid dosage forms, such as capsules or tablets.

In one embodiment of the invention, the free base composition coated beads may additionally be coated with an enteric coating. In yet another embodiment, a seal coating may be applied to the drug containing beads prior to the application of the enteric coating. After the coatings are applied, the beads may be admixed with sufficient quantities of pharmaceutically necessary tableting excipients. Pharmaceutical tableting excipients include but are not limited to a lubricant, disintegrant, binder, glidant and/or inert diluent. The tablets thus formulated may further be coated with a film-coating.

In processing the free base composition granules into tablets, as disclosed above, the granules may be admixed with at least one pharmaceutically necessary excipient and compressed into the tablets. Pharmaceutically acceptable excipients include but are not limited to a lubricant, a disintegrant, a binder, a glidant and/or an inert diluent.

The invention is also directed to a method of treating human patients, comprising administering to human patients an effective amounts of the free base composition formulations prepared in accordance with the invention.

The invention is further related to a method of treating hypertension, angina and migraines in humans comprising orally administering an effective dose of metoprolol free base formulations prepared in accordance with the invention.

### DETAILED DISCLOSURE OF THE INVENTION

The present invention provides a process for preparing free base, e.g., metoprolol free base, solid oral dosage forms, where the process comprises combining an acid addition salt of a pharmaceutical compound, at least one solvent and a sufficient amount of at least one base to convert the acid addition salt of the pharmaceutical to a free base of the pharmaceutical, to form a free base composition of the pharmaceutical compound. Preferably, the base is added in a sufficient amount to raise the pH above the pK value (in order to effect conversion to the free base) without the addition of excess base or an excessive quantity of excess base. An excessive quantity of excess base would be an amount which is capable of causing irritation to a patient. In certain embodiments, the pH of the free base composition is preferably 10.8±1.0, most preferably ±0.5.

In an embodiment of the invention, the free base composition may be prepared by dissolving an acid addition salt of a pharmaceutical in a basic solution (e.g. sodium hydroxide in an organic/aqueous cosolvent). Additional sodium hydroxide may be added to ensure that the acid addition salt is substantially converted to the free base.

In another embodiment, water and acetone are combined to form an aqueous-organic co-solvent. A pharmaceutically active acid-addition salt is then dissolved in the co-solvent. The co-solvent system is then basified using a weak base, e.g., by adding a solution of an organic or inorganic basic compound to the co-solvent system. While the base is being added, the acid addition salt undergoes conversion to the free base. Ion certain embodiments, the organic free base is completely dissolved by the organic solvent and the water dissolves the functional acid and the resulting solution contains a completely dissolved organic free base in an aqueous-organic solvent. In certain embodiments, water in the solvent system allows for processing temperatures to be raised higher than if the solvent were strictly organic. In further embodiments utilizing water, surfactants are utilized to facilitate the processing of the organic free base into a solid dosage form.

In certain embodiments, the free base composition may be sprayed onto a pharmaceutically acceptable carrier, and the resulting mixture may then be processed to obtain free base composition tablets.

In one embodiment, the pharmaceutically acceptable carrier comprises a plurality of particles of a material that is an inert diluent, and the free base composition is sprayed onto the carrier and dried to produce free base composition granules. In another embodiment of the invention, a binder may also be combined with the free base composition and the pharmaceutically acceptable carrier.

In certain embodiments of the invention, the free base composition is sprayed onto the pharmaceutically acceptable carrier in a fluid bed processor with or without a Wurster apparatus or similar apparatus. In alternative embodiments, the free base composition can be sprayed onto the carrier using a high shear granulator.

In certain embodiments, the free base composition can be diluted, e.g., with an organic solvent such as isopropyl alcohol or acetone, before it is sprayed onto the carrier.

The base used in the present invention can be any pharmaceutically acceptable organic or inorganic base or alkaline agent such as sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide, tribasic phosphate, potassium phosphate and mixtures thereof. A preferred base is sodium hydroxide. In certain embodiments, the at least one base includes at least one basic amino acid, e.g., L-arginine.

Examples of pharmaceutically acceptable carriers include, but are not limited to, calcium phosphate dihydrate, calcium sulfate dihydrate, microcrystalline cellulose (e.g., Celspheres), cellulose derivatives, dextrose, lactose, anhydrous lactose, spray-dried lactose, lactose monohydrate, mannitol, starches, sorbitol and sucrose. Further examples of the carrier include hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, and suitable mixtures of the foregoing. Preferred pharmaceutically acceptable carriers include anhydrous lactose or microcrystalline cellulose.

In certain embodiments, optional pharmaceutical excipients are added to the free base composition granules in the process of formulating the granules into tablets. Such pharmaceutical excipients may include but are not limited to a lubricant, disintegrant, binder, glidant and/or diluent.

Examples of lubricants include magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, sodium stearate, glycerol monostearate, and zinc stearate.

Suitable disintegrants include crospovidone, alginates, cellulose and its derivatives, clays, polyvinylpyrrolidone, polysaccharides, such as corn and potato starch, dextrins, croscarmellose sodium, and sugars. Disintegrants, when used in the formnulation, facilitates disintegration when the tablet contacts water in the gastrointestinal tract.

Binders, when added to the formulation, promote granulation and/or promote cohesive compact during the direct compression into tablets. Examples of binders include acacia, cellulose derivatives, gelatin, glucose, guar gum, polyvinylpyrrolidone, sodium alginate and alginate derivatives, sorbitol, and starch. Binders also include hydrophillic cellulose gums, such as methylcellulose and carboxymethylcellulose, and xanthan gum.

Examples of glidants include but are not limited to corn starch, silica derivatives, and talc.

Examples of inert diluents can include, but are not limited to, calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose. Further examples of the carrier include hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, and suitable mixtures of the foregoing. A preferred pharmaceutically acceptable carrier is microcrystalline cellulose.

The tablet cores described above may be coated with an enteric coating to obtain delayed-release tablets that remain intact in the stomach and release the active ingredient in the intestine. Suitable enteric coating may comprise cellulose acetate phthalate, polyvinyl acetate phthalate, acrylic resins such as Eudragit L, shellac, cellulose acetate butyrate, hydroxypropyl methylcellulose phthalate or combinations thereof.

Additional materials suitable for use in the enteric coating include phthalates including cellulose acetyl phthalate, cellulose triacetyl phthalate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, and polyvinyl acetate phthalate. The enteric materials are discussed in Remington's Pharmaceutical Sciences, 17th Ed., page 1637 (1985).

The enteric coating may be applied by press coating, molding, spraying, dipping and/or air-suspension or air tumbling procedures. A preferred method of applying the enteric coating is by pan coating, where the enteric coating is applied by spraying the enteric composition onto the tablet cores accompanied by tumbling in a rotating pan. The enteric coating material may be applied to the tablet cores by employing solvents, including an organic, aqueous or a mixture of an organic and aqueous solvent. Examplary solvents suitable in applying the enteric coating include an alcohol, ketone, ester, ether, aliphatic hydrocarbon, halogenated solvents, cycloaliphatic solvents, aromatic, heterocyclic, aqueous solvents, and mixtures thereof.

In certain embodiments, the enteric coating comprises a plasticizer. Suitable plasticizers for use in the present invention include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. Preferred plasticizers include triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 15% of the plasticizer can be used based upon the total weight of the coating.

In a preferred embodiment, the enteric coating comprises cellulose acetate phthalate and diethyl phthalate.

In accordance with the invention, the free base tablet cores may further be coated with a seal coating. In a preferred embodiment, the seal coating occurs between the tablet core and the enteric coating. The seal coating may comprise a hydrophilic polymer. Examples include but are not limited to hydroxypropyl cellulose, hydroxypropylmethylcellulose, methoxypropyl cellulose, hydroxypropylisopropylcellulose, hydroxypropylpentylcellulose, hydroxypropylhexylcellulose and any mixtures thereof.

The seal coating, like the enteric coating, may be applied by press coating, molding, spraying, dipping and/or air-suspension or air tumbling procedures. A preferred method of applying the seal coating is by pan coating, where the seal coating is applied by spraying it onto the tablet cores accompanied by tumbling in a rotating pan. The seal coating material may be applied to the tablets as a suspension by employing solvents, e.g., an organic, aqueous, or a mixture of an organic and aqueous solvent. Examplary solvents suitable in applying the seal coating include aqueous-based solutions, an alcohol, ketone, ester, ether, aliphatic hydrocarbon, halogenated solvents, cycloaliphatic solvents, aromatic, heterocyclic, aqueous solvents, and mixtures thereof. In a preferred embodiment, the seal coating comprises hydroxypropyl cellulose and hydroxypropylmethylcellulose, and is delivered as a suspension using ethanol as a solvent.

The free base tablets may be overcoated with a pharmaceutically acceptable film coating, e.g., for aesthetic purposes (e.g., including a colorant), for stability purposes (e.g., coated with a moisture barrier), for taste-masking purposes, etc. For example, the tablets may be overcoated with a film coating, preferably containing a pigment and a barrier agent, such as hydroxypropylmethycellulose and/or a polymethylmethacrylate. An example of a suitable material which may be used for such overcoating is hydroxypropylmethylcellulose (e.g., Opadry®, commercially available from Colorcon, West Point, Pa.). In a preferred embodiment, an overcoating is applied to the tablets that have already been coated with a seal coating and an enteric coating. The overcoat may be applied using a coating pan or a fluidized bed, and may be applied by using a solvent, preferably an aqueous solvent.

The final product is optionally subjected to a polishing step to improve the appearance of the final product and also to facilitate the manipulation of the formulation post manufacture. For example, the slippery nature of the polished dosage form aids in filling printer carrier bars with the formulation and facilitates final packaging of the product. Suitable polishing agents are polyethylene glycols of differing molecular weight or mixtures thereof, talc, surfactants (e.g., Brij types, Myrj types, glycerol mono-stearate and poloxamers), fatty alcohols (e.g., stearyl alcohol, cetyl alcohol, lauryl alcohol and myristyl alcohol) and waxes (e.g., camauba wax, candelilla wax and white wax). Preferably, polyethylene glycols having molecular weight of 3,000-20,000 are employed.

In certain embodiments of the present invention, the pharmaceutically acceptable carrier onto which the free base composition is sprayed comprises a plurality of inert beads, e.g., sugar beads such as Celspheres. The free base coated beads thus obtained may be coated with an enteric coating. The beads may also be coated with a seal coating, preferably the seal coating being applied before the enteric coating. The suitable enteric coating and the seal coating materials are set forth above.

The free base coated beads may be formulated into solid oral dosage forms. For example, the beads made be formulated into tablets by admixing them with sufficient quantities of a pharmaceutically necessary tableting excipient and compressing the resulting mixture. The pharmaceutically necessary tableting excipient is selected from the group consisting of a lubricant, a disintegrant, a binder, a glidant, an inert diluent and mixtures thereof. Suitable tableting excipients are set forth above.

In certain preferred embodiments, the present invention provides a process for preparing free base delayed-release tablets. The process comprises preparing a free base composition by combining an acid addition salt of a pharmaceutically active agent an organic/aqueous cosolvent and a base, e.g., sodium hydroxide, the bases being added in sufficient amount to ensure conversion of the acid addition salt of the pharmaceutical to a free base, and spraying the free base composition onto a pharmaceutically acceptable diluent, processing the resulting mixture to obtain free base granules, and processing the granules to obtain tablet cores. An enteric coating is applied to the free base tablet cores to produce free base delayed-release tablets. Preferably, the delayed-release tablet further comprises a seal coating, applied between the core and the enteric coating. Suitable material for the seal coating and the enteric coating, as well as the procedures for application of these coatings, are set forth above.

In a preferred embodiment, the processing of the free base granules to obtain tablets comprises drying and then screening the free base granules, and admixing the screened free base granules with pharmaceutically necessary excipients and compressing the resulting mixture into tablets. The pharmaceutically acceptable excipients are selected from the group consisting of a lubricant, a disintegrant, a binder, a glidant, an inert diluent and mixtures thereof. Examples of suitable excipients are listed above.

In certain embodiments, the free base composition is diluted with isopropyl alcohol before it is sprayed onto anhydrous lactose in a fluid bed processor with a Wurster apparatus at product temperature of, e.g., 42-48° C. and a spray rate of, e.g., 40-80 ml/min to form granules. The granules are sized through an appropriate sized screen, e.g., a 16 mesh screen. The sized granules are blended with crospovidone, anhydrous lactose, colloidal silicon dioxide and magnesium stearate and compressed into tablets. The tablets are coated with a seal coating in a coating pan with a suspension of hydroxypropylmethylcellulose, hydroxypropylethylcellulose, hydroxypropyl cellulose and magnesium stearate in ethanol. An enteric coating is then applied, also in a coating pan. The enteric coating comprises cellulose acetate phthalate and diethyl phthalate in isopropyl alcohol and acetone. As an optional final step, the enteric coated tablet is film coated and subjected to a polishing step.

The acid addition salts of the pharmaceutically active agent include such salts as the hydrochloride, hydrobromide, sulfate, hydrogen sulfate, phosphate, hydrogen phosphate, dihydrogenphosphate, acetate, besylate, succinate, tartrate, fumarate, citrate, methanesulfonate (mesylate) and p-toluenesulfonate (tosylate) salts.

In preferred embodiments, the active agent is metoprolol and the acid addition salt is preferably metoprolol tartrate, metoprolol fumarate or metoprolol succinate. The most preferred salt is metoprolol succinate.

The solvent can be an aqueous solvent, an organic solvent, or an aqueous/organic cosolvent. Examples of suitable organic solvents which can be utilized include but are not limited to methanol, ethanol, propanol (including both n-propanol and i-propanol), butanol (including n-butanol, I-butanol, and s-butanol), toluene, benzene supercritical liquid CO₂, chloroform, methylene chloride, acetonitrile, ketones (e.g. dimethylketone, methylethylketone, and diethylketone), dimethylformamide, dimethylsulfoxide, esters (a non-limiting example being ethyl acetate), ethers (non-limiting examples being diethylether, dipropylether), 1,4-dioxane, tetrahydrofuran, pentanes, hexanes, heptanes, trichloroethene, and/or suitable mixtures thereof. In preferred embodiments, the organic solvent is selected from the group consisting of acetone, methanol, ethanol, isopropyl alcohol and mixtures thereof.

In certain embodiments utilizing aqueous/organic cosolvents, the ratio of organic solvent to water can be about 1:9 to about 9:1, preferably about 1:4 to about 4:1, more preferably about 1:2 to about 2:1 and most preferably about 1:1.5.

In certain embodiments, it is preferable to use an aqueous/organic cosolvent.

### EXAMPLES

The following examples illustrate various aspects of the present invention. They are not to be construed to limit the claims in any manner whatsoever.

### Example 1

### Preparation of Metoprolol Free Base

To an aqueous-organic co-solvent solution of 16.446 kg purified water and 11.513 kg acetone, 3.544 kg metoprolol succinate and 0.638 kg of L-arginine were dissolved with stirring. A sodium hydroxide solution 0.494 kg sodium hydroxide and 1.978 kg purified water was than added to the metoprolol succinate solution with stirring to form a metoprolol free base solution.

### Example 2

### Preparation of Metoprolol Free Base Granules

A top spray Glatt fluidized bed granulator was set up with the following parameters. One skilled in the art would understand that other parameters can be used.

TABLE 1
Spray Glatt fluidized bed granulator parameters

| a. | Nozzle configuration: | Top Spray | |
|---|---|---|---|
| b. | Nozzle tip: | 1.8 | mm |
| c. | Screen size: | 200 | mesh |
| d. | Shaking interval: | 30 | seconds |
| e. | Shaking duration: | 1 | second |
| f. | Atomization pressure: | 1.0-3.0 | bar |
| g. | Inlet air temperature: | 35° C.-80° C. | |
| h. | Pump rate: | 80-150 | ml/min |
| i. | Tubing size: | 24 | mm |

The fluidized bed granulator was preconditioned such that the inlet and bed temperature were about 40° C. and 2.263 kg of microcrystalline cellulose were charged into the fluidized bed granulator. The granulation cycle was then initiated using the parameter described in Table 1 as a guideline and the metoprolol free base solution from Example 1 was sprayed onto the 2.263 kg of microcrystalline cellulose in the fluidized bed granulator. After granulation was completed, the product was dried for about 30 minutes at product temperature between about 30° C. and 40° C. The granules were unloaded and placed in an oven at 40 C. for about 12 to 18 hours or until the LOD (loss-on-dry) was less than 3%. The LOD machine was set for 10 minutes at 105° C. and a final moisture result of 1.325% was obtained. The dried granules were then discharged from the oven and a weight of 6.24 kg of dried granules was obtained.

The dried granules were than passed through a Comil equipped with a # 1143 size stainless steel screen and a 0.15 spacer at medium speed (speed 2695 RPM (40%)) and into a container with a final weight of 6.11 kg remaining. The percent of the theoretical yield was 96.6%.

### Example 3

### Blending and Tabletin

Metroprolol Succinate Granules, 1.887 kg (as prepared in Example 2), 0.012 kg of colloidal silicon dioxide, 2.000 kg of hydroxypropyl methylcellulose (Methocel K100M) and 0.071 kg of lactose monohydrate (spray-dried) were charged into a blender after passing them through a #1143 screen at medium speed. The above ingredients were blended for about twenty minutes at 32 rpm. Stearic acid, 0.030 kg, was passed through a #30 mesh and then charged into the blender and blending continued for five more minutes at 32 rpm. The blend was then checked for quality control testing and weighed. A final weight of 3.936 kg was obtained. A percent yield of 99.7%.

The blend was then compressed into tablets by using a tablet press to yield metoprolol base tablets.

### Example 4

### Preparation of Film Coated Tablets

The metoprolol tablets as prepared in example 3 were coated with Opadry Clear. A solution of Opadry Clear was prepared by adding 59.25 g of Opadry Clear into a mixture of into a mixture of 490 g of isopropyl alcohol and 210 g of purified water while stirring at high rpm until the big lumps disperse. Stirring was continued at low rpm until a clear solution was obtained. The total mixing time was about 60 minutes. Then 19.54 g of sodium chloride was added with continued stirring until the sodium chloride powder was dissolved to prepare an aqueous coating solution. The resulting aqueous coating solution was then sprayed onto the metoprolol free base tablets and the tablets were process using an O'HARA LABCOATII pan coater (O'HARA Technologies, Research Triangle Park, N.C.) to form film coated tablets.

### Example 5 and 6

In accordance with the above examples, metoprolol free base formulations were prepared with the ingredients as set forth below:

| | Example 5 | Example 6 |
|---|---|---|
| Granules | | |
| metoprolol succinate | 33.44% | 50.35% |
| L-Arginine | 16.72% | 9.06% |
| NaOH | 4.68% | 7.02% |
| MCC 101 | 33.45% | 33.57% |
| S 100 | 11.71% | — |
| Uncoated Tablet | | |
| metoprolol granules | 84.0% | 49.01% |
| K4M | 7.5% | — |
| K100M | 7.5% | 50.24% |
| GMS | 1% | — |
| Cab-O-Sil | — | 0.35% |
| Mg. Stearate | — | 0.4% |

An Opadry Clear/NaCl coating in a ration of 3:1 was applied to Example 5.

### Example 7

### Biostudies

Data from clinical trials of 200 mg formulations of Examples 5 and 6 as compared to Toprol XL® 100 mg are set forth below:

| Parameter | Toprol ®XL | Example 5 | G-Ratio |
|---|---|---|---|
| Cₘₐₓ (Fast) | 48.38 | 122.09 | 2.607 |
| AUC₍₀₋₄₈₎ (Fast) | 1001.01 | 1246.10 | 1.355 |
| T ₘₐₓ (Fast) | 8.0 | 7.00 | 0.917 |
| Cₘₐₓ (Fed) | 74.04 | 220.07 | 2.390 |
| AUC₍₀₋₄₈₎ (Fed) | 1908.15 | 2887.79 | 1.449 |
| T ₘₐₓ (Fed) | 7.43 | 5.71 | 0.783 |

| Parameter | Toprol ®XL | Example 6 | G-Ratio |
|---|---|---|---|
| Cₘₐₓ (Fast) | 56.38 | 65.72 | 1.244 |
| AUC₍₀₋₄₈₎ (Fast) | 1166.64 | 975.72 | 0.948 |
| T ₘₐₓ (Fast) | 7.00 | 6.44 | 0.951 |
| Cₘₐₓ (Fed) | 65.54 | 120.58 | 1.809 |
| AUC₍₀₋₄₈₎ (Fed) | 1260.67 | 1687.20 | 1.132 |
| T ₘₐₓ (Fed) | 6.38 | 6.5 | 1.109 |

### Example 8

### Biostudies

Data from clinical trials of 200 mg formulations made in accordance with the above examples as compared to Toprol XL® 200 mg are set forth below:

| Parameter | Toprol ®XL | Formulation A | % Ratio |
|---|---|---|---|
| Cₘₐₓ (Fast) | 249.08 | 232.42 | 93.31 |
| AUC₍₀₋ₜ₎ (Fast) | 5165.41 | 4484.45 | 86.82 |
| T ₘₐₓ (Fast) | 7.75 | 4.83 | 89.91 |

| Parameter | Toprol ®XL | Formulation B | % Ratio |
|---|---|---|---|
| Cₘₐₓ (Fast) | 249.08 | 271.58 | 109.03 |
| AUC₍₀₋ₜ₎ (Fast) | 5165.41 | 4842.48 | 93.75 |
| T ₘₐₓ (Fast) | 7.75 | 7.33 | 91.44 |

### Example 8

A co-solvent is prepared by combining 1346.59 g H₂0 with 942.62 g Acetone. Dissolve 290.22 g of metoprolol succinate in the co-solvent. After the metoprolol succinate is completely dissolved, add 52.26 g of L-Arginine. The solution is clear.

40.50 g sodium hydroxide is then dissolved in 162.00 g H₂0. The sodium hydroxide solution is then slowly added to the co-solvent containing the drug. The co-solvent is stirred during the addition of the sodium hydroxide solution.

The resulting free base solution is clear with no visible signs of phase separation or precipitate. The final solution comprises acetone:water in a ratio of 1:1.6.

3.84 g polysorbate 80 and 6.78 g hydroxypropyl methylcellulose is the added to the free base solution. This solution is then sprayed onto 206.40 g of sugar spheres producing metoprolol active pellets.

The active pellets may then be formed into tablets, placed inside capsules, or further processed with additional coatings. These coating may include but would not be limited to: controlled release, sustained release, delayed release, or chronotherapy type coatings.

1. A process for preparing a free base composition of a pharmaceutical compound comprising:
combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound.

2. The process of claim 1, comprising
(a) dissolving the acid addition salt of the pharmaceutical compound in the at least one solvent; and
(b) adding the at least one base in a sufficient amount to substantially convert the acid addition salt of the pharmaceutical to a free base of the pharmaceutical, to form a free base composition of the pharmaceutical compound.

3. The process of claim 1 wherein said at least one base includes a basic amino acid.

4. The process of claim 1, wherein the solvent is a selected from the group consisting of an organic solvent, an aqueous solvent and an organic-aqueous co-solvent.

5. The process of claim 4, wherein the solvent is an organic-aqueous co-solvent.

6. The process of claim 3, wherein the basic amino acid is selected from the group consisting of tribasic phosphate, potassium phosphate and L-Arginine.

7. The process of claim 1, wherein said acid addition salt is an acid addition salt of metoprolol.

8. A process according to claim 7, wherein the metoprolol acid addition salt is selected from the group consisting of metoprolol succinate, metoprolol tartrate, metoprolol fumarate and combinations thereof.

9. A process according to claim 8, wherein the metoprolol salt is metoprolol succinate.

10. A process according to claim 1, wherein the organic solvent is selected from the group consisting of isopropyl alcohol, methanol, ethanol, acetone and combinations thereof.

11. A process according to claim 10, wherein the organic solvent is acetone.

12. A process according to claim 1, wherein the base is selected from the group consisting of sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide and combinations thereof.

13. A process according to claim 12, wherein the base is sodium hydroxide.

14-33. (canceled)

34. A method of treating hypertension comprising administering an effective dose of a formulation made according to the process of claim 7.

35. A method of treating angina pectoris comprising administering an effective dose of a formulation made according to the process of claim 7.

36. A method of treating migraines comprising administering an effective dose of a formulation made according to the process of claim 7.

37. (canceled)

38. A method of increasing the bioavailability of a pharmaceutical compound comprising:
combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound;
incorporating the free base composition into a dosage form in a therapeutically effective amount, wherein the bioavailability of the free base composition is greater than the bioavailability achieved by an equivalent amount of the acid addition salt of the pharmaceutical compound.

39. (canceled)

40. A method of utilizing reduced amounts of a pharmaceutical compound comprising:
combining an acid addition salt of a pharmaceutical compound, at least one solvent and a sufficient amount of at least one base to substantially convert the acid addition salt of the pharmaceutical to a free base of the pharmaceutical, to form a free base composition of the pharmaceutical compound;
incorporating the free base composition into a dosage form in an amount to achieve a therapeutically effective AUC measurement of the free base composition, wherein the amount of the free base composition is less than an amount of the acid addition salt of the pharmaceutical compound to achieve the same AUC.

41. (canceled)

42. A method of treating a patient comprising:
administering a free base pharmaceutically active agent in an amount to achieve a therapeutically effective AUC measurement of the active agent, wherein the amount of the free base active agent is less than an amount of an acid addition salt of the active agent to achieve the same AUC measurement.

43. A pharmaceutical dosage form comprising:
an active drug in free base form;
said dosage form providing bioavailability for the active drug at least substantially equal to the bioavailability provided by a dosage form comprising the base equivalent amount of an acid addition salt of the active drug component.
